(19) United States
(12) Patent Application Publication
(10) Pub. No.: US 2010/0035978 A1
(43) Pub. Date: Feb. 11, 2010

(54) COMBINATION OF CANNABINOIDS FOR THE TREATMENT OF PERIPHERAL NEUROPATHIC PAIN

(75) Inventors: Geoffrey Guy, Wiltshire (GB); Stephen Wright, Wiltshire (GB); Philip Robson, Wiltshire (GB)

Correspondence Address: WOLF GREENFIELD & SACKS, P.C., 600 ATLANTIC AVENUE, BOSTON, MA, 02210-2206, US

(73) Assignee: GW Pharma Limited, Salisbury, Wiltshire (GB)

(21) Appl. No.: 12/084,454

(22) PCT Filed: Oct. 31, 2006
(86) PCT No.: PCT/GB2006/004063

§ 371 Date: Aug. 28, 2009

(30) Foreign Application Priority Data
Nov. 1, 2005 (GB) 0522311.0

(51) Int. Cl. A61K 31/352; A61P 25/02; A61P 25/20

(52) U.S. Cl. 514/454

(57) ABSTRACT

The present invention relates to the use of a combination of cannabinoids in the treatment of neuropathic pain, in particular peripheral neuropathic pain. A combination of cannabidiol (CBD) and delta-9-tetrahydrocannabinol (THC) may be used, wherein the ratio of CBD:THC by weight is between 10:1 and 1:10.

### FIELD OF THE INVENTION

The present invention relates to the use of a combination of cannabinoids for the treatment of neuropathic pain, in particular peripheral neuropathic pain characterised by mechanical allodynia, more preferably when the peripheral neuropathic pain is characterised by post-herpetic neuralgia. Preferably the combination of cannabinoids are cannabidiol (CBD) and delta-9-tetrahydrocannabinol (THC). More preferably the cannabinoids are in a predefined ratio by weight of approximately 1:1 of CBD to THC.

### BACKGROUND TO THE INVENTION

Pain is one of the most common reasons for a patient to seek medical care and in consequence pain, results in a tremendous number of lost work days per year.

Neuropathic pain is caused by abnormalities in the nerves, spinal cord or brain and is a chronic type of non-malignant pain with an estimated prevalence of over 1% of the population. Optimising pain relief in these patients is crucial in helping a patient regain control of his or her life.

The most common cause of neuropathic pain is injury or dysfunction of nerves. Injury or dysfunction of peripheral nerves or nerves descending from the spinal cord results in disinhibition of nerve impulses at the spinal cord which in consequence results in pain. Neuropathic pain can also be centrally mediated, rather than peripheral, in conditions such as spinal cord injury and multiple sclerosis.

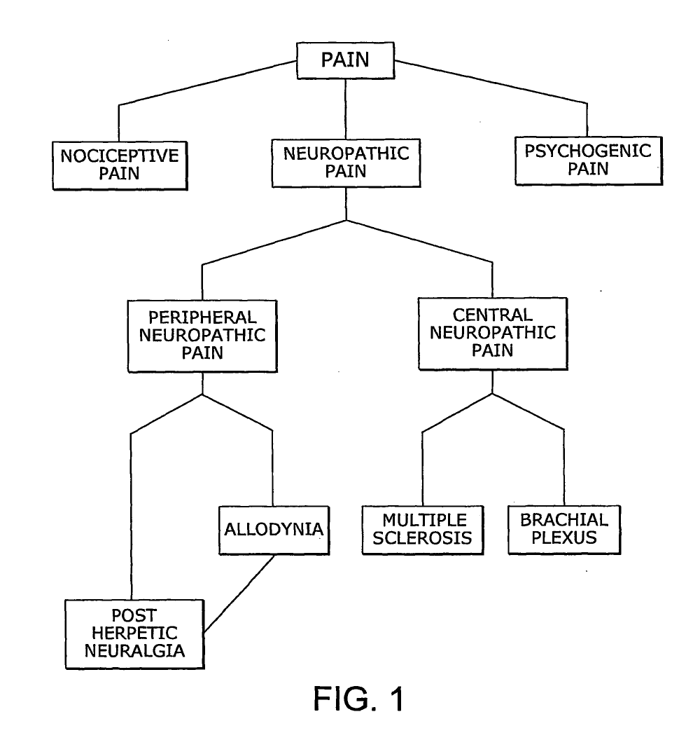

FIG. 1 describes the different types of pain and how certain types of diseases such as allodynia and multiple sclerosis are classified by these different types of pain.

Pain can be caused by stimulation of the sensory nerve endings called nociceptors, such as occurs after injury or surgery. This type of pain is called nociceptive pain. Pain signals are transmitted by the nociceptors to the brain. Often the pain is localised, constant and has an aching or throbbing quality. Once the damage to the tissue heals the pain usually resolves. Treatment with opioids usually resolves nociceptive pain.

Another type of pain is psychogenic pain, this is a pain disorder that is associated with psychological factors. Some types of mental or emotional problems can cause pain. They can also increase or prolong pain. Headaches, muscle pains, back pain, and stomach pains are some of the most common types of psychogenic pain.

People with this pain disorder actually have real pain. The diagnosis is made when organic causes of pain are ruled out.

A different class of pain is neuropathic pain and is the result of an injury or malfunction of the peripheral nervous system or the central nervous system. The pain may be triggered by an injury but not necessarily by an injury of the nervous system itself. Neuropathic pain is frequently chronic and is often less responsive to treatment with opioids, but may respond to treatment with anticonvulsant or antidepressant drugs.

Neuropathic pain can be divided into two classes; peripheral neuropathic pain and central neuropathic pain depending on whether the peripheral or central nervous system is affected.

FIG. 1 details examples of the types of central neuropathic pain such as multiple sclerosis and brachial plexus which result in pain caused by damage or inflammation of the central nerves. Damage or inflammation of the peripheral nerves is often characterised by conditions such as allodynia and post-herpetic neuralgia.

Patients with peripheral neuropathic pain often experience pain which feels like a burning or electrical pain, whereas others describe their pain as feeling like extreme cold or pins and needles.

The pain may be worsened by activity or by wearing clothes over the affected area. The pain may also follow a daily pattern which may mean it is worse at certain times of the day.

Allodynia is a type of peripheral neuropathic pain. This is a painful response to a typically non-painful stimulus, for example brushing the affected area with a fingertip. The pain tends to increase with repeated stimulation and may spread from the affected area. Allodynic pain can be evoked in response to mechanical, thermal (cold or heat) or chemical low or high intensity stimuli applied either statically or dynamically to skin, joints, bone, muscle or viscera. It is thought that the presence of allodynic pain is a more suitable means of grouping patients suffering from peripheral neuropathic pain than by the specific disease that led to the neuropathic pain.

Post-herpetic neuralgia results from a complication of shingles which is caused by the herpes zoster virus. Patients suffering from post-herpetic neuralgia have inflammation in their nerve tissue. Pain is felt as a constant deep aching or burning sensation and can be sharp or intermittent. It may also be felt as a hypersensitivity to touch or cold. Very often patients find that the pain is debilitating.

As it can be seen post-herpetic neuralgia is a type of allodynic pain as well as being a type of peripheral neuropathic pain.

Other types of peripheral neuropathic pain include hereditary disorders such as Charcot-Marie Tooth disease and Friedreich's ataxia; systemic or metabolic disorders such as diabetic neuropathy, vitamin B12 deficiency, alcoholic neuropathy, uremia or cancer; infectious or inflammatory conditions such as AIDS, hepatitis, Guillain-Barre Syndrome and sarcoidosis; or exposure to toxic chemicals.

It is clear that patients that suffer from neuropathic pain can have their quality of life greatly affected by it. The pain can interfere with work and social activities as well as with the amount and quality of sleep that a patient experiences. A successful treatment for the relief of neuropathic pain should improve both the amount of pain that the patient is experiencing as well as improving the patient's quality of life.

Non-pharmaceutical methods of treating neuropathic pain include transcutaneous electrical nerve stimulation (TENS) and acupuncture.

The use of pharmaceuticals is the most common treatment for neuropathic pain. These include topical creams applied directly to the site of pain. Analgesics, antidepressants and anticonvulsants are the other drug classes generally in use. The drug carbamezepine which is an anticonvulsant is currently the only FDA approved drug which has an indication for neuropathic pain. It has been suggested in post-marketing studies that there is a five- to eight-fold increase in the risk of blood dyscrasias in patients taking carbamezepine. In 7% of patients there has been shown to be a 25% decrease in their white blood cell count, this usually reverses within the first 4 months of therapy.

The use of cannabis as a medicine has long been known and during the 19^{th} Century preparations of cannabis were recommended as a hypnotic sedative which were useful for the treatment of hysteria, delirium, epilepsy, nervous insomnia, migraine, pain and dysmenorrhoea.

Until recent times the administration of cannabis to a patient could only be achieved by preparation of cannabis by decoction in ethanol, which could then be swallowed or by the patient inhaling the vapours of cannabis by smoking the dried plant material. Recent methods have sought to find new ways to deliver cannabinoids to a patient including those which bypass the stomach and the associated first pass effect of the liver which can remove up to 90% of the active ingested dose and avoid the patient having to inhale unhealthy tars and associated carcinogens into their lungs.

Such dosage forms include administering the cannabinoids to the sublingual or buccal mucosae, inhalation of a cannabinoid vapour by vaporisation or nebulisation, enemas or solid dosage forms such as gels, capsules, tablets, pastilles and lozenges.

The use of different ratios of cannabinoids such as THC or CBD or their propyl variants, tetrahydrocannabinovarin (THCV) and cannabidivarin (CBDV), in the treatment of different diseases and conditions has previously been described by the applicant in their International patent application WO02/064109.

Specific ratios of THC and CBD or THCV and CBDV were reported to have been useful in the treatment or management of specific diseases or medical conditions.

Formulations containing specific, defined ratios of cannabinoids may be formulated from pure, synthetic cannabinoids or from extracts derived from the cannabis plant in combination with pharmaceutical carriers and excipients.

Peripheral neuropathic pain is often associated with a diverse and complex set of pain stimuli and are difficult to treat effectively as the response to treatment is unpredictable.

Surprisingly the applicants have found that administration of a medicament that contains a combination of the cannabinoids cannabidiol (CBD) and delta-9-tetrahydrocannabinol (THC) to patients with peripheral neuropathic pain results in a significant improvement of their 11-point Numerical Rating Scale (NRS) scores. Also most of the patients reported an improvement in their pain even though they were taking their existing medication throughout the trial.

### SUMMARY OF INVENTION

According to the first aspect of the present invention there is provided the use of a combination of cannabinoids cannabidiol (CBD) and delta-9-tetrahydrocannabinol (THC) in the manufacture of a pharmaceutical formulation for use in the treatment of peripheral neuropathic pain, wherein the ratio of CBD:THC by weight is between 10:1 and 1:10.

Preferably the peripheral neuropathic pain is characterised by allodynia.

Preferably the peripheral neuropathic pain is characterised by post-herpetic neuralgia.

In a second aspect of the present invention there is provided the use of a combination of cannabinoids cannabidiol (CBD) and delta-9-tetrahydrocannabinol (THC) in the manufacture of a pharmaceutical formulation for use in the treatment of sleep disturbance caused by peripheral neuropathic pain, wherein the ratio of CBD:THC by weight is between 10:1 and 1:10.

Preferably the ratio of CBD:THC by weight is between 5:1 and 1:5. More preferably the ratio of CBD:THC by weight is between 2:1 and 1:2. Most preferably the ratio of CBD:THC by weight is substantially 1:1, more particularly still the ratio of CBD:THC by weight is 0.93:1.

Favourably the cannabinoids are packaged for delivery in a titratable dosage form.

The cannabinoid CBD may be administered separately, simultaneously or sequentially to the cannabinoid THC.

The administration of a combination of cannabinoids such as THC and CBD to a patient could either be at the same time, wherein the cannabinoids would be contained in the same formulation. The cannabinoids could also be administered at separate times for example; a formulation containing CBD could be administered to a patient at a fixed time prior to a formulation containing THC in order to ameliorate some of the side effects of THC, which CBD is known to improve or vice versa. The two cannabinoids could also be administered consecutively to a patient if required.

The term “titrate” is defined as meaning that the patient is provided with a medication that is in such a form that smaller doses than the unit dose can be taken.

A “unit dose” is herein defined as a maximum dose of medication that can be taken at any one time or within a specified dosage period such as 3 hours.

Titration of doses are beneficial to the patient as they are able to take smaller numbers of doses of the medication until the drug is efficacious. It is understandable that not all patients will require exactly the same dose of medication, for example patients of a larger build or faster metabolism may require a higher dose than that required by a patient that is of a smaller build. Different patients may also present with different degrees of complaints and as such may require larger or smaller doses in order to treat the complaint effectively. The benefits of a titratable dosage form over dosage forms where smaller, incremental doses are difficult to take, are therefore evident.

Unit dose ranges are preferably in the range of between 5 and 25 mg of each cannabinoid CBD and THC, more preferably in the range of 10 to 20 mg of each cannabinoid, preferably in the range of 12 to 14 mg of each cannabinoid more preferably still in the range of 12.5 to 13.5 mg of each cannabinoid.

Preferably the maximum daily dosage dose of medicament is less than or equal to 120 mg CBD and less than or equal to 130 mg THC.

Preferably the pharmaceutical formulations are packaged for delivery such that delivery is targeted to an area selected from one or more of the following: sublingual; buccal; oral; rectal, nasal; and the pulmonary system.

More preferably the pharmaceutical formulations are in the form selected from one or more of the following: gel; gel spray; tablet; liquid; capsule and for vaporisation.

Additionally the pharmaceutical formulation further comprises one or more carrier solvents. Preferably the carrier solvents are ethanol and/or propylene glycol. More preferably the ratio of ethanol to propylene glycol is between 4:1 and 1:4. More preferably still the ratio is substantially 1:1.

Preferably the cannabinoids are present as a cannabis based medicine extract (CBME).

More preferably the combination of cannabinoids comprises:
a cannabis based medicinal extract which comprises THC at more than 90% of the total cannabinoid content in the extract; and
a cannabis based medicinal extract which comprises CBD at more than 90% of the total cannabinoid content in the extract.

Alternatively the combination of cannabinoids are substantially pure, preferably the combination of cannabinoids are synthetic.

In one embodiment the CBME are produced by extraction with supercritical or subcritical CO₂. In an alternative embodiment the CBME are produced by extraction from plant material by volatilisation with a heated gas. Preferably the CBME contain all of the naturally occurring cannabinoids in the plant material. Alternatively synthetic or highly purified isolates of the cannabinoids can be used.

According to a third aspect of the present invention there is provided the use of a combination of cannabinoids cannabidiol (CBD) and delta-9-tetrahydrocannabinol (THC), in the manufacture of a pharmaceutical formulation for use in the treatment of peripheral neuropathic pain, wherein the ratio of CBD:THC by weight is between 10:1 and 1:10, wherein the cannabinoids are administered in combination with one or more other medicinal substances.

Preferably the combination of cannabinoids are administered in addition to one or more analgesic drugs.

More preferably still the combination of cannabinoids are administered in addition to one or more opiate or opiate related drugs.

Opiate or opiate related drugs include but are not limited to drugs chemically related to morphine and also non-related structures which act at the same receptors in the brain.

Preferably the combination of cannabinoids are administered in addition to one or more anticonvulsant drugs.

Preferably the combination of cannabinoids are administered in addition to one or more antidepressant drugs.

The term “in combination” refers to administration of the cannabinoids at the same time and in the same formulation as the opiate or opiate related drug.

The term “in addition to” refers to administration of the cannabinoids to patient who is already being administered opiate or opiate related drugs.

More preferably the combination of cannabinoids are administered separately, simultaneously or sequentially to the one or more other drugs.

The different therapeutic classes of medications that are useful to be used in addition to the combination of cannabinoids include but are not limited to: natural opium alkaloids, anti-epileptics, non-selective monoamine reuptake inhibitors, opioids, anilides, diphenylpropylamine derivatives, acetic acid derivatives and related substances, platelet aggregation inhibitors excluding heparin, carboxamide derivatives, propionic acid derivatives, salicylic acid derivatives, local anaesthetics, non-steroidal anti-inflammatory or anti-rheumatic compounds, coxibs, topical non-steroidal anti-inflammatory compounds, opium alkaloids and derivatives, anaesthetics for topical use, drugs used in opioid dependence, hydantoin derivatives, oripavine derivatives, phenylpiperidine derivatives.

The term “approximately equal” is used to refer to ratios of cannabinoids which are in the range of between 0.9:1 to 1:0.9 (THC:CBD). Additionally the term “1:1” is taken herein to refer to approximately equal amounts of cannabinoids.

Certain aspects of this invention are further described, by way of example only, with reference to the accompanying drawings in which:

FIG. 1 shows a diagram describing of the different types of pain;

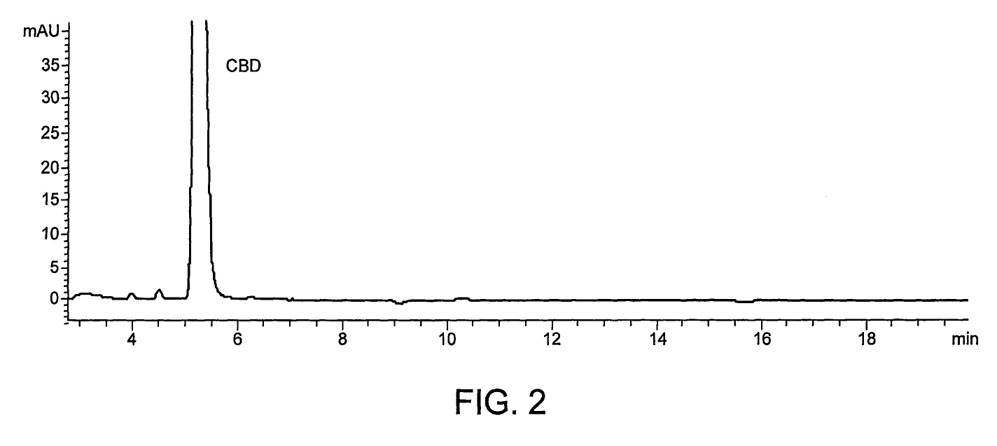

FIG. 2 shows an HPLC chromatographic profile which characterises a CBD-containing cannabis based medicine extract;

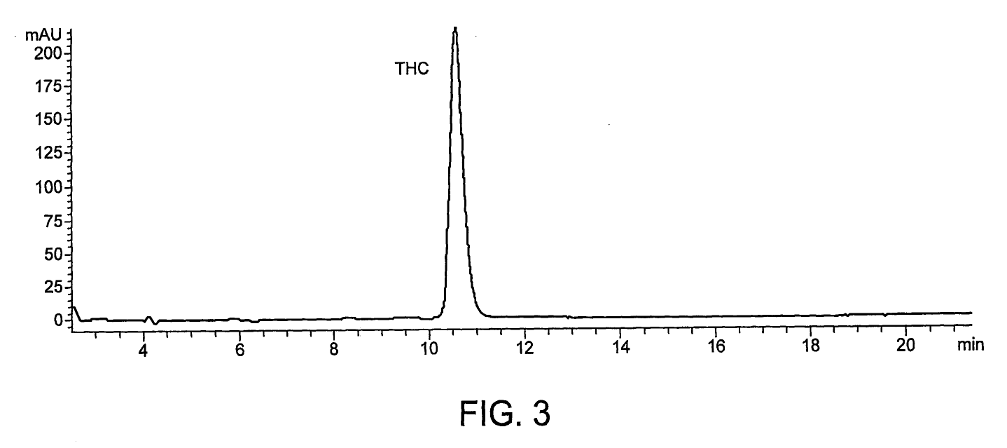

FIG. 3 shows an HPLC chromatographic profile which characterises a THC-containing cannabis based medicine extract; and

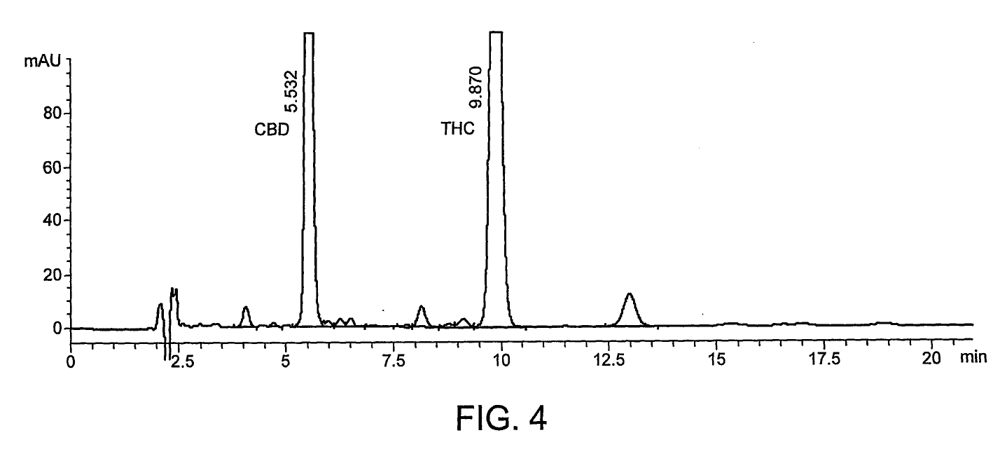

FIG. 4 shows an HPLC chromatographic profile which characterises a cannabis based medicine extract comprising substantially equal quantities of CBD and THC.

### SPECIFIC DESCRIPTION

A cannabis based medicine extract (CBME) was prepared as outlined in Example 1 and contained approximately equal amounts of the cannabinoids THC and CBD and this was administered to patients with peripheral neuropathic pain characterised with allodynia.

A six week double blind, randomised, parallel group, placebo-controlled study of different cannabis based medicine extracts (CBME) was undertaken. The test articles that were studied were CBME THC:CBD (1:1) and matching placebo.

The study population were male or female patients aged 18 years or above, who have peripheral neuropathic pain characterised by allodynia. For inclusion in the study patients were required to have a history of at least 6 months duration of pain due to a clinically identifiable peripheral nerve lesion and were able to demonstrate mechanical allodynia as well as impairment of sensation within the territory of affected nerves and evidences of sensory derangement on clinical examination.

Some of the patients with peripheral neuropathic pain characterised by allodynia had the condition post-herpetic neuralgia. The data for these patients was examined as a discrete group as well as part of the wider study group in order that the effectiveness of the study medication could be evaluated in this specific disease group.

A baseline pain score of at least 4 on the Numerical rating Scale (NRS) for spontaneous pain on at least four of seven days in the baseline week was also required for eligibility of the study. Also required was a stable medication regimen of analgesics for at least two weeks prior to the study commencing. The study medication was to be maintained concomitantly with the patient's existing medication throughout the study period.

A summary of all medications taken by patients in the trial are listed below:

| | No. of | |
|---|---|---|
| | patients in | No. of patients |
| Patient's Existing | THC:CBD (1:1) | in Placebo |
| Medication | group (%) | group (%) |
| Natural opium | 20 (31.7) | 32 (51.6) |
| alkaloids | | |
| Anti-epileptics | 20 (31.7) | 18 (29.0) |
| Non-selective | 11 (17.5) | 19 (30.6) |
| monoamine reuptake | | |
| inhibitors | | |
| Opioids | 11 (17.5) | 8 (12.9) |
| Anilides | 9 14.3) | 8 (12.9) |
| Diphenylpropylamine | 9 (14.3) | 6 (9.7) |
| derivatives | | |
| Acetic acid | 4 (6.3) | 6 (9.7) |
| derivatives and | | |
| related substances | | |
| Platelet aggregation | 8 (12.7) | 2 (3.2) |
| inhibitors excluding | | |
| heparin | | |
| Carboxamide | 5 (7.9) | 3 (4.8) |
| derivatives | | |
| Propionic acid | 3 (4.8) | 4 (6.5) |
| derivatives | | |
| Salicylic acid | 2 (3.2) | 3 (4.8) |
| derivatives | | |
| Local anaesthetics | 2 (3.2) | 2 (3.2) |
| Non-steroidal anti- | 1 (1.6) | 2 (3.2) |
| inflammatory or anti- | | |
| rheumatic compounds | | |
| Coxibs | 2 (3.2) | 1 (1.6) |
| Topical non-steroidal | 1 (1.6) | 1 (1.6) |
| anti-inflammatory | | |
| compounds | | |
| Opium alkaloids and | 1 (1.6) | 1 (1.6) |
| derivatives | | |
| Anaesthetics for | 1 (1.6) | 0 |
| topical use | | |
| Drugs used in opioid | 1 (1.6) | 0 |
| dependence | | |
| Hydantoin derivatives | 1 (1.6) | 0 |
| Oripavine derivatives | 1 (1.6) | 0 |
| Phenylpiperidine | 1 (1.6) | 0 |
| derivatives | | |

The primary objective of the study was to evaluate the efficacy of the 1:1 THC:CBD study medication compared with the placebo in relieving peripheral neuropathic pain. The change from baseline in peripheral neuropathic pain severity was measured using an 11-point NRS scores.

The secondary objectives of the study were to evaluate the effect of the 1:1 THC:CBD study medication compared with placebo on:
Qualitative-aspects of pain as reported in the Neuropathic Pain Scales (NPS).
The physical and Psychological effects of peripheral neuropathic pain using measures of sleep disturbance, the Pain Disability Index (PDI) and a 12 item General Health Questionnaire (GHQ-12)
The subject's cognitive function using the Brief Repeatable Battery of Neuropsychological tests (BRB-N).
The subject's perception of change in peripheral neuropathic pain severity and allodynic pain compared with before study entry, using 7-point Patient's Global Impression of Change (PGIC) scales.

The patient's tolerability of the study medication was also evaluated using the adverse event profile, electrocardiogram traces, clinical laboratory testing and vital signs.

Surprisingly the cannabis based medicine extract containing approximately equal quantities of THC and CBD was shown to be a well-tolerated adjunct therapy in patients with neuropathic pain refractory to existing analgesic medication. In particular in patients suffering from post-herpetic neuralgia.

A clinically significant difference was obtained with the 1:1 THC:CBD study medication and this is especially important in the patients of this study who are considered to be unlikely to respond to treatment.

Additionally patients that were administered the CBME containing approximately equal amounts of THC and CBD were shown to have an improved PDI score and improved relief from sleep disturbance. It was also shown from the results of the BRB-N that the self-reported improvements in pain and function found in this study were an analgesic effect and did not result from mood enhancement.

The features of the invention are illustrated further by reference to the following examples:

### Example 1

### Preparation of Cannabis Based Medicine Extracts (CBME)

Medicinal cannabis was produced and prepared with reference to the method disclosed in WO 02/064109 (Example 15). The resulting plant material was processed as described in the flow chart below. The process of manufacture of a High THC or High CBD cannabis based medicine extract is described.

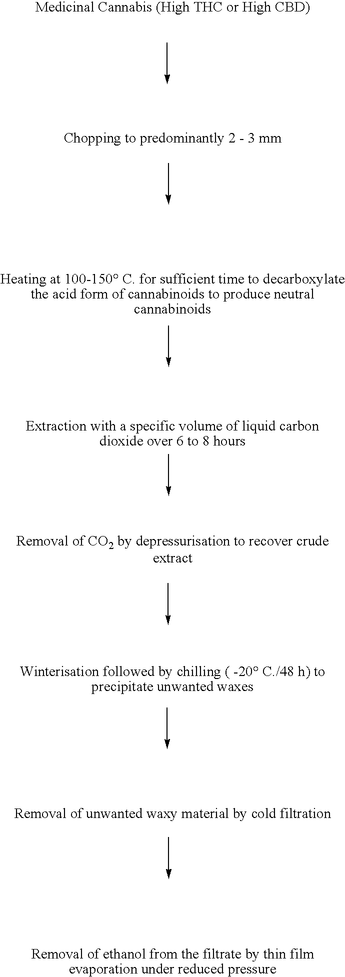

The resulting extract is referred to as a cannabis based medicinal drug extract and is also classified as a Botanical Drug Substance according to the US Food and Drug Administration Guidance for Industry Botanical Drug Products.

The quantity of cannabinoid in the CBME can be accurately assessed by way of measurement by HPLC with reference to the method disclosed in WO 02/064109 (Example 16).

An example of an HPLC chromatogram of a CBD-containing CBME produced using a high CBD medicinal cannabis plant extracted with CO₂ is shown in FIG. 2. An example of an HPLC chromatogram of a THC-containing CBME produced using a high THC medicinal cannabis plant extracted with CO₂ is shown in FIG. 3. An example of an HPLC chromatogram containing the relevant ratios of THC and CBD CBMEs is shown in FIG. 4.

### Example 2

### Evaluation of the Efficacy of a Cannabis-Based Medicine Extract (CBME) Containing Approximately Equal Ratios of Delta-9-Tetrahydrocannabinol (THC) and Cannabidiol (CBD) in Relieving Peripheral Neuropathic Pain after Five Weeks of Treatment, Using Change in Baseline in Peripheral Neuropathic Pain Severity Measured Using an 11-Point Numerical Rating Scale (NRS) Scores

A six week double blind, randomised, parallel group, placebo-controlled study of different cannabis based medicine extracts (CBME) was undertaken. The test articles that were studied were CBME THC:CBD (1:1) and matching placebo.

The cannabis based medicine extracts contained delta-9-tetrahydrocannabinol (THC) at a concentration of 27 mg/ml and cannabidiol (CBD) at a concentration of 25 mg/ml in ethanol:propylene glycol (50:50) excipient. The CBME was presented in a pump action spray where each activation delivers 100 μl of spray, containing THC (2.7 mg) and CBD (2.5 mg).

The subjects in the study were randomised equally to either the cannabis based medicine extracts or placebo. The placebo matched the appearance, smell, colour and taste of the active formulation, but contained no active components, the excipients were ethanol:propylene glycol (50:50) excipient. Again the placebo was presented in a pump action spray where each activation delivers 100 μl of spray.

The maximum dose of study medication that was allowed to be taken was 8 sprays at any one time or within any 3 hour interval, with a maximum of 48 sprays within any 24 hour interval.

It should be noted that the terms “1:1 THC:CBD” or “equal amounts of THC:CBD” refer to approximately equal amounts of the two cannabinoids.

At the screening visit the patients were assessed for compliance with the inclusion or exclusion criteria and advised of the study requirements.

Dosing was introduced under clinical supervision at week 0 with monitoring of safely and tolerability and introduction of intoxication scales. During self-titration patients were shown how to record their dosing in a patient diary.

The primary outcome measure was a change from baseline on a numerical rating scale (NRS) of intensity of pain where 0=“no pain” and 10=“worst pain possible”. The baseline severity score was defined as the mean of all diary entries from Day −7 to Day −1. The end of treatment score was defined as the mean of all diary entries during the last seven days of the study or the last three days if the patient withdrew due to worsening pain or lack of efficacy.

The secondary outcome measures included the neuropathic pain scale, tests for mechanical allodynia, a four-step verbal rating scale for sleep disturbance, the pain disability index, the general health questionnaire, assessment of the short-term changes in mental health, social dysfunction and somatic symptoms, cognitive functions using the brief repeatable battery of neuropsychological tests, patients global impression of change and an intoxication visual analogue scale.

The testing for allodynia was carried out twice. At the screening visit the patients identified the most painful area within the affected territory which was recorded by the investigator to ensure that the repeat testing was carried out on the same area.

Mechanical dynamic allodynia was assessed by the investigator stroking the skin over the affected area five times with a standardised brush designed specifically for sensory testing at 5 second intervals and recording the pain severity on a 0-10 point scale after each brush, 5 times. The mean pain severity was compared between treatment groups in the same way as for the primary outcome measure.

Punctate allodynia score was determined using an in-house built pressure algometer comprising a strain gauge connected to a metal filament with a diameter of 1 mm. The filament was pressed perpendicularly against the skin and the reading taken as soon as the patient recorded a sensation of pain. The pressure reading and the intensity of the invoked pain were recorded.

Results:

Some of the data collated from this study is described below.

Analysis of Efficacy of the 1:1 THC:CBD Study Medication Compared with the Placebo in Relieving Peripheral Neuropathic Pain in the Intention to Treat Study Population.

The mean baseline intensity of reported pain in both the study medicine group and the placebo group were in the severe range, these were 7.29 and 7.21 respectively.

In the group given the study medication there was an adjusted mean decrease in NRS pain score from baseline to the end of treatment of 1.48 points (20.3%). For the placebo group there was an adjusted mean decrease of 0.52 points (7.2%). The treatment difference of 0.96 points was significantly in favour of the study medication the 1:1 THC:CBD.

Table 1 details the results obtained in the Intention to Treat population.

TABLE 1

| | | THC:CBD | |
|---|---|---|---|
| | | (27 mg/ml:25 mg/ml) | Placebo |
| | | (N = 63) | (N = 62) |
| Baseline | Mean | 7.29 | 7.21 |
| | Std Dev | 1.384 | 1.463 |
| | Median | 7.20 | 7.08 |
| | Minimum | 4.0 | 4.0 |
| | Maximum | 10.0 | 10.0 |
| Week 1 | Mean | 6.38 | 6.91 |
| | Std Dev | 1.832 | 1.735 |
| | Median | 6.29 | 7.07 |
| | Minimum | 2.2 | 3.0 |
| | Maximum | 9.9 | 10.0 |
| Week 1 - | Mean | −0.88 | −0.30 |
| change | Std Dev | 1.540 | 0.856 |
| from | Median | −0.37 | −0.25 |
| baseline | Minimum | −5.1 | −3.0 |
| | Maximum | 2.2 | 1.9 |
| Week 2 | Mean | 6.17 | 6.56 |
| | Std Dev | 2.215 | 2.159 |
| | Median | 6.29 | 7.00 |
| | Minimum | 1.2 | 0.9 |
| | Maximum | 10. | 10.0 |
| Week 2 - | Mean | −1.14 | −0.67 |
| change | Std Dev | 1.646 | 1.287 |
| from | Median | −0.67 | −0.33 |
| baseline | Minimum | −5.0 | −5.4 |
| | Maximum | 1.7 | 2.4 |
| Week 3 | Mean | 5.52 | 6.55 |
| | Std Dev | 2.564 | 2.278 |
| | Median | 5.86 | 7.14 |
| | Minimum | 0.5 | 1.0 |
| | Maximum | 10.0 | 10.0 |
| Week 3 - | Mean | −1.76 | −0.69 |
| change | Std Dev | 1.997 | 1.245 |
| from | Median | −1.00 | −0.47 |
| baseline | Minimum | −7.1 | −4.0 |
| | Maximum | 1.3 | 2.4 |
| Week 4 | Mean | 5.50 | 6.57 |
| | Std Dev | 2.623 | 2.192 |
| | Median | 5.57 | 6.86 |
| | Minimum | 0.0 | 0.4 |
| | Maximum | 10.0 | 10.0 |
| Week 4 - | Mean | −1.77 | −0.64 |
| change | Std Dev | 2.124 | 1.352 |
| from | Median | −0.94 | −0.37 |
| baseline | Minimum | −7.9 | −4.1 |
| | Maximum | 1.2 | 2.4 |
| Week 5 | Mean | 5.37 | 6.51 |
| | Std Dev | 2.615 | 2.206 |
| | Median | 5.93 | 6.77 |
| | Minimum | 0.0 | 0.8 |
| | Maximum | 10.0 | 10.0 |
| Week 5 - | Mean | −1.85 | −0.70 |
| change | Std Dev | 2.207 | 1.324 |
| from | Median | −1.30 | −0.23 |
| baseline | Minimum | −7.9 | −4.9 |
| | Maximum | 1.2 | 1.2 |

Scores range from 0 (No pain) to 10 (Worst possible pain).

The baseline is the average of all available data recorded during the 7 days immediately prior to the randomisation visit.

Statistical analysis of this data is shown in Table 2.

Table 2 details the Analysis of Covariance of the mean 11-point NRS pain scores in the intention to treat (ITT) population.

TABLE 2

| | | Difference from | | p- |
|---|---|---|---|---|
| | Mean | placebo | 95% CI | value |
| THC:CBD | −1.48 | −0.96 | [−1.59, −0.32] | 0.004 |
| (27 mg/ml:25 mg/ml) | | | | |
| Placebo | −0.52 | — | — | — |

Table 3 details the reduction from baseline in the 11-point NRS pain scores in the intention to treat (ITT) population.

TABLE 3

| Reduction in | THC:CBD | |
|---|---|---|
| baseline | (27 mg/ml:25 mg/ml) | Placebo |
| ≧30% | 16 (26.2%) | 9 (14.5%) |
| <30% | 45 (73.8%) | 53 (85.5%) |
| ≧50% | 12 (19.7%) | 5 (8.1%) |
| <50% | 49 (80.3%) | 57 (91.9%) |

Table 4 details the treatment differences in the 30% and 50% responders.

TABLE 4

| Reduction in | Treatment | |
|---|---|---|
| baseline | difference | Odds Ratio |
| 30% | 11.71 | 2.09 |
| 50% | 11.61 | 2.79 |

The treatment difference value is calculated as the percentage of responders who reported a 30 or 50% reduction in baseline score in the study medication group minus the percentage of responders who reported a 30 or 50% reduction in baseline score in the placebo group. A positive treatment difference indicates an improvement with the 1:1 THC:CBD over the placebo.

The data shown above illustrates that the study medication which contained approximately equal amounts of THC and CBD resulted in a greater change from the baseline in pain scores when compared to the study medication which contained THC alone. As such the statistical analysis data demonstrates that the 1:1 THC:CBD is shown statistically to be more efficacious than the placebo.

Analysis of Efficacy of the 1:1 THC:CBD Study Medication Compared with the Placebo in Relieving Peripheral Neuropathic Pain in the Per-Protocol Study Population.

Table 5 details the results obtained in the per-protocol population.

TABLE 5

| | | THC:CBD | |
|---|---|---|---|
| | | (27 mg/ml:25 mg/ml) | Placebo |
| | | (N = 63) | (N = 62) |
| Baseline | Mean | 7.34 | 7.27 |
| | Std Dev | 1.361 | 1.484 |
| | Median | 7.39 | 7.17 |
| | Minimum | 5.0 | 4.0 |
| | Maximum | 10.0 | 10.0 |
| Week 1 | Mean | 6.34 | 6.89 |
| | Std Dev | 1.960 | 1.770 |
| | Median | 6.29 | 7.00 |
| | Minimum | 2.2 | 3.0 |
| | Maximum | 9.9 | 10.0 |
| Week 1 - | Mean | −0.99 | −0.38 |
| change | Std Dev | 1.601 | 0.807 |
| from | Median | −0.57 | −0.29 |
| baseline | Minimum | −5.1 | −3.0 |
| | Maximum | 2.2 | 1.1 |
| Week 2 | Mean | 5.93 | 6.55 |
| | Std Dev | 2.221 | 2.171 |
| | Median | 5.79 | 7.00 |
| | Minimum | 1.2 | 0.9 |
| | Maximum | 10.0 | 10.0 |
| Week 2 - | Mean | −1.41 | −0.72 |
| change | Std Dev | 1.622 | 1.220 |
| from | Median | −0.98 | −0.33 |
| baseline | Minimum | −5.0 | −5.4 |
| | Maximum | 1.0 | 1.3 |
| Week 3 | Mean | 5.38 | 6.62 |
| | Std Dev | 2.630 | 2.187 |
| | Median | 5.79 | 7.14 |
| | Minimum | 0.5 | 1.0 |
| | Maximum | 10.0 | 10.0 |
| Week 3 - | Mean | −1.95 | −0.61 |
| change | Std Dev | 2.151 | 1.236 |
| from | Median | −1.30 | −0.33 |
| baseline | Minimum | −7.9 | −4.1 |
| | Maximum | 1.2 | 1.7 |
| Week 4 | Mean | 5.42 | 6.64 |
| | Std Dev | 2.698 | 2.122 |
| | Median | 5.50 | 6.93 |
| | Minimum | 0.0 | 0.4 |
| | Maximum | 10.0 | 10.0 |
| Week 4 - | Mean | −1.92 | −0.61 |
| change | Std Dev | 2.151 | 1.236 |
| from | Median | −1.30 | −0.33 |
| baseline | Minimum | −7.9 | −4.1 |
| | Maximum | 1.2 | 1.7 |
| Week 5 | Mean | 5.30 | 6.53 |
| | Std Dev | 2.697 | 2.157 |
| | Median | 5.86 | 6.83 |
| | Minimum | 0.0 | 0.8 |
| | Maximum | 10.0 | 10.0 |
| Week 5 - | Mean | −1.98 | −0.65 |
| change | Std Dev | 2.257 | 1.323 |
| from | Median | −1.31 | −0.20 |
| baseline | Minimum | −7.9 | −4.9 |
| | Maximum | 1.2 | 1.2 |

Scores range from 0 (No pain) to 10 (Worst possible pain).

The baseline is the average of all available data recorded during the 7 days immediately prior to the randomisation visit.

Statistical analysis of this data is shown in Table 6.

Table 6 details the Analysis of Covariance of the mean 11-point NRS pain scores in the per-protocol population.

TABLE 6

| | | Difference from | | p- |
|---|---|---|---|---|
| | Mean | placebo | 95% CI | value |
| THC:CBD | −1.96 | −1.42 | [−2.10, −0.74] | <0.001 |
| (27 mg/ml:25 | | | | |
| mg/ml) | | | | |
| Placebo | −0.54 | — | — | — |

Table 7 details the reduction from baseline in the 11-point NRS pain scores in the per-protocol population.

TABLE 7

| Reduction in | THC:CBD | |
|---|---|---|
| baseline | (27 mg/ml:25 mg/ml) | Placebo |
| ≧30% | 16 (33.3%) | 7 (12.3%) |
| <30% | 32 (66.7%) | 50 (87.7%) |
| ≧50% | 12 (25.0%) | 4 (7.0%) |
| <50% | 36 (75.0%) | 53 (93.0%) |

Table 8 details the treatment differences in the 30% and 50% responders.

| Reduction in | Treatment | |
|---|---|---|
| baseline | difference | Odds Ratio |
| 30% | 21.05 | 3.57 |
| 50% | 17.98 | 4.42 |

The treatment difference value is calculated as the percentage of responders who reported a 30 or 50% reduction in baseline score in the study medication group 10 minus the percentage of responders who reported a 30 or 50% reduction in baseline score in the placebo group. A positive treatment difference indicates an improvement with the 1:1 THC:CBD over the placebo.

The data shown above confirms that shown by the ITT population in that the study medication which contained approximately equal amounts of THC and CBD resulted in a greater change from the baseline in pain scores when compared to the study medication which contained THC alone. As such the statistical analysis data demonstrates that the 1:1 THC:CBD is shown statistically to be more efficacious than the placebo.

Analysis of Efficacy of the 1:1 THC:CBD Study Medication in the Secondary Outcomes of the Study.

a) Neuropathic Pain Scale (NPS)

Table 9 shows a summary of the Neuropathic Pain Scale Total Scores in the Intention to Treat Population.

TABLE 9

| | | THC:CBD | |
|---|---|---|---|
| | | (27 mg/ml:25 | |
| | | mg/ml) | Placebo |
| Baseline | Mean | 61.1 | 62.4 |
| (Visit 2) | Std Dev | 12.93 | 13.68 |
| | Median | 63.0 | 60.5 |
| | Minimum | 30 | 34 |
| | Maximum | 90 | 93 |
| Visit 4 | Mean | 50.9 | 60.4 |
| | Std Dev | 21.53 | 16.76 |
| | Median | 56.0 | 61.5 |
| | Minimum | 0 | 17 |
| | Maximum | 94 | 93 |
| Change | Mean | −9.7 | −2.0 |
| from | Std Dev | 19.35 | 12.14 |
| baseline | Median | −5.0 | −0.5 |
| | Minimum | −69 | −34 |
| | Maximum | 24 | 31 |

The data detailed above shows that there was a greater change from baseline in the group treated with the 1:1 THC:CBD than with placebo. Statistical analysis was performed on the data and a p-value of 0.007 was obtained showing a statistically significant improvement of symptoms in the study medication treated group.

b) Pain Disability Index (PDI)

The pain disability index showed improvement with the study medication when compared to the placebo. Overall in the seven functional areas assessed by the PDI there was a statistically significant finding (p=0.003) in favour of the 1:1 THC:CBD group.

One area of the PDI resulted in a dramatic improvement. This was the area of sleep disturbance. Table 10 details the sleep disturbance scores recorded by patients in the Intention to Treat population. Sleep disturbance was scored using a system of the number of times woken in the previous night due to symptoms where 1 none, 2=once, 3=twice and 4=more than twice.

TABLE 10

| | | THC:CBD | |
|---|---|---|---|
| | | (27 mg/ml:25 | |
| | | mg/ml) | Placebo |
| Baseline | Mean | 2.99 | 2.97 |
| | Std Dev | 0.838 | 0.939 |
| | Median | 3.14 | 3.24 |
| | Minimum | 1.0 | 1.0 |
| | Maximum | 4.0 | 4.0 |
| Week 1 | Mean | 2.30 | 2.74 |
| | Std Dev | 0.905 | 0.885 |
| | Median | 2.15 | 2.71 |
| | Minimum | 1.0 | 1.0 |
| | Maximum | 4.0 | 4.0 |
| Change | Mean | −0.65 | −0.23 |
| from | Std Dev | 0.632 | 0.512 |
| baseline | Median | −0.58 | −0.14 |
| | Minimum | −2.4 | −1.9 |
| | Maximum | 0.4 | 1.5 |
| Week 2 | Mean | 2.17 | 2.57 |
| | Std Dev | 0.896 | 0.975 |
| | Median | 2.00 | 2.43 |
| | Minimum | 1.0 | 1.0 |
| | Maximum | 4.0 | 4.0 |
| Change | Mean | −0.78 | −0.39 |
| from | Std Dev | 0.707 | 0.671 |
| baseline | Median | −0.68 | −0.29 |
| | Minimum | −2.5 | −3.0 |
| | Maximum | 0.6 | 0.9 |
| Week 3 | Mean | 2.07 | 2.60 |
| | Std Dev | 0.928 | 0.994 |
| | Median | 2.00 | 2.64 |
| | Minimum | 1.0 | 1.0 |
| | Maximum | 4.0 | 4.0 |
| Change | Mean | −0.85 | −0.38 |
| from | Std Dev | 0.749 | 0.650 |
| baseline | Median | −0.71 | −0.19 |
| | Minimum | −2.6 | −3.0 |
| | Maximum | 0.4 | 1.1 |
| Week 4 | Mean | 2.04 | 2.65 |
| | Std Dev | 0.888 | 0.981 |
| | Median | 1.86 | 2.71 |
| | Minimum | 1.0 | 1.0 |
| | Maximum | 4.0 | 4.0 |
| Change | Mean | −0.88 | −0.36 |
| from | Std Dev | 0.738 | 0.668 |
| baseline | Median | −0.76 | −0.14 |
| | Minimum | −2.6 | −3.0 |
| | Maximum | 0.4 | 0.9 |
| Week 5 | Mean | 2.06 | 2.63 |
| | Std Dev | 0.931 | 1.026 |
| | Median | 1.86 | 2.57 |
| | Minimum | 1.0 | 1.0 |
| | Maximum | 4.0 | 4.0 |
| Change | Mean | −0.92 | −0.39 |
| from | Std Dev | 0.771 | 0.718 |
| baseline | Median | −0.77 | −0.14 |
| | Minimum | −2.6 | −3.0 |
| | Maximum | 0.3 | 1.2 |

As it can be seen from the data detailed in Table 10 there was a greater mean change in baseline score for the group treated with the THC:CBD medication than with the placebo. Statistical analysis on the data resulted in a statistically significant value of p=0.001 in favour of the 1:1 THC:CBD study medication.

The data from the other secondary endpoints all demonstrated an improvement in patients treated with the 1:1 THC:CBD in comparison with the placebo.

Analysis of Efficacy of the 1:1 THC:CBD Study Medication Compared with the Placebo in Relieving Peripheral Neuropathic Pain in the Post-Herpetic Neuralgia Study Population.

The mean baseline intensity of reported pain in both the study medicine group and the placebo group were in the severe range, these were 7.21 and 7.66 respectively.

In the group given the study medication there was a mean decrease in NRS pain score from baseline to the end of treatment of −0.72 points. This was a decrease in the pain scores of 10%.

For the placebo group there was an adjusted mean decrease of 0.45 points. This was an increase in the pain scores of 17%.

The treatment difference was therefore significantly in favour of the 1:1 THC:CBD study medication.

Table 11 details the results obtained in the individual patients in the study medication group.

TABLE 11

| | | End of | |
|---|---|---|---|
| Patient | | Treatment | Change from |
| Number | Baseline | Period | Baseline |
| 134 | 8.0 | 5.6 | −2.4 |
| 150 | 7.3 | 7.3 | 0 |
| 180 | 6.3 | 4.7 | −1.6 |
| 215 | 6.8 | 5.9 | −1.0 |
| 116 | 9.9 | 10 | 0.1 |
| 192 | 6.4 | 6.3 | −0.1 |
| 205 | 8 | 8 | 0 |
| 181 | 6.4 | 5.7 | −0.7 |
| 198 | 7 | 6.7 | −0.3 |
| 204 | 6 | 4.8 | −1.2 |

Table 12 details the results obtained in the individual patients in the placebo group.

TABLE 12

| | | End of | Change |
|---|---|---|---|
| Patient | | Treatment | from |
| Number | Baseline | Period | Baseline |
| 138 | 9.4 | 9.6 | 0.2 |
| 147 | 8.3 | 9.0 | 0.7 |
| 135 | 8.0 | 8.0 | 0 |
| 194 | 5.9 | 6.9 | 1.0 |
| 111 | 6.9 | 8.3 | 1.5 |
| 158 | 6.3 | 5.7 | −0.6 |
| 163 | 8.8 | 9.3 | 0.5 |

Scores range from 0 (No pain) to 10 (Worst possible pain). A negative change from the baseline score indicates an improvement of pain.

Statistical analysis of these data is shown in Table 13.

Table 13 details the Analysis of Covariance of the mean 11-point NRS pain scores in the intention to treat (ITT) population.

TABLE 13

| | | Difference |
|---|---|---|
| | | from |
| | Mean | placebo |
| THC:CBD | −0.72 | −0.26 |
| (27 mg/ml:25 mg/ml) | | |
| Placebo | 0.46 | — |

The data shown above illustrates that the study medication which contained approximately equal amounts of THC and CBD resulted in a greater change from the baseline in pain scores when compared to the study medication which contained THC alone. As such the statistical analysis data demonstrates that the 1:1 THC:CBD is shown statistically to be more efficacious than the placebo.

It can therefore be concluded that a medication that contains approximately equal amounts of THC and CBD offers a new treatment option in the treatment of patients with neuropathic pain, in particular patients with neuropathic pain characterised by allodynia, more particularly in patients suffering from post herpetic neuralgia.

1.-24. (canceled)

25. A method of treating peripheral neuropathic pain in a human patient comprising administering to a patient in need thereof a therapeutically effective amount of a combination of cannabinoids cannabidiol (CBD) and delta-9-tetrahydrocannabinol (THC), wherein the ratio of CBD:THC by weight is between 10:1 and 1:10.

26. A method of treating peripheral neuropathic pain characterised by allodynia in a human patient comprising administering to a patient in need thereof a therapeutically effective amount of a combination of cannabinoids cannabidiol (CBD) and delta-9-tetrahydrocannabinol (THC), wherein the ratio of CBD:THC by weight is between 10:1 and 1:10.

27. A method of treating peripheral neuropathic pain characterised by post-herpetic neuralgia in a human patient comprising administering to a patient in need thereof a therapeutically effective amount of a combination of cannabinoids cannabidiol (CBD) and delta-9-tetrahydrocannabinol (THC), wherein the ratio of CBD:THC by weight is between 10:1 and 1:10.

28. A method of treating sleep disturbance caused by peripheral neuropathic pain in a human patient comprising administering to a patient in need thereof a therapeutically effective amount of a combination of a combination of cannabinoids cannabidiol (CBD) and delta-9-tetrahydrocannabinol (THC), wherein the ratio of CBD:THC by weight is between 10:1 and 1:10.

29. (canceled)

30. The method as claimed in claim 25, wherein the ratio of CBD:THC by weight is between 5:1 and 1:5.

31. The method as claimed in claim 25, wherein the ratio of CBD:THC by weight is between 2:1 and 1:2.

32. The method as claimed in claim 25, wherein the ratio of CBD:THC by weight is substantially 1:1.

33. The method as claimed in claim 32, wherein the ratio of CBD:THC by weight is 0.93:1.

34. The method as claimed in claim 25, wherein the cannabinoids are packaged for delivery in a titratable dosage form.

35. The method as claimed in claim 25, wherein the cannabinoid CBD is administered separately, simultaneously or sequentially to the cannabinoid THC.

36. The method as claimed in claim 25, wherein a unit dose taken by a patient is in the range of 5-25 mg of each cannabinoid.

37. The method as claimed in claim 25, wherein the maximum daily dosage dose of each cannabinoid is less than or equal to 120 mg of CBD and less than or equal to 130 mg of THC.

38. The method as claimed in claim 25, wherein the pharmaceutical formulations are packaged for delivery such that delivery is targeted to an area selected from the group: sublingual; buccal; oral; rectal, nasal; and the pulmonary system.

39. The method as claimed in claim 38, wherein the pharmaceutical formulations are in the form selected from the group: gel; gel spray; tablet; liquid; capsule and for vaporisation.

40. The method as claimed in claim 25, wherein the cannabinoids are present as a cannabis based medicine extract (CBME).

41. The method as claimed in claim 25, wherein the combination of cannabinoids comprises:
a) a cannabis based medicinal extract which comprises THC at more than 90% of the total cannabinoid content in the extract; and
b) a cannabis based medicinal extract which comprises CBD at more than 90% of the total cannabinoid content in the extract.

42. The method as claimed in claim 25, wherein the cannabinoids are substantially pure.

43. The method as claimed in claim 25, wherein the cannabinoids are synthetic.

44. The method as claimed in claim 25, wherein the cannabinoids are administered in combination with one or more other medicinal substances.

45. The method as claimed in claim 44, wherein the cannabinoids are administered in addition to one or more analgesic drugs, one or more opiate or opiate related drugs, one or more anticonvulsant drugs and/or one or more antidepressant drugs.

46. The method as claimed in claim 44, wherein the cannabinoids are administered separately, simultaneously or sequentially to the one or more other drugs.

47. The method as claimed in claim 26, wherein the ratio of CBD:THC by weight is between 5:1 and 1:5.

48. The method as claimed in claim 26, wherein the ratio of CBD:THC by weight is between 2:1 and 1:2.

49. The method as claimed in claim 26, wherein the ratio of CBD:THC by weight is substantially 1:1.

50. The method as claimed in claim 49, wherein the ratio of CBD:THC by weight is 0.93:1.

51. The method as claimed in claim 26, wherein the cannabinoids are packaged for delivery in a titratable dosage form.

52. The method as claimed in claim 26, wherein the cannabinoid CBD is administered separately, simultaneously or sequentially to the cannabinoid THC.

53. The method as claimed in claim 26, wherein a unit dose taken by a patient is in the range of 5-25 mg of each cannabinoid.

54. The method as claimed in claim 26, wherein the maximum daily dosage dose of each cannabinoid is less than or equal to 120 mg of CBD and less than or equal to 130 mg of THC.

55. The method as claimed in claim 26, wherein the pharmaceutical formulations are packaged for delivery such that delivery is targeted to an area selected from the group: sublingual; buccal; oral; rectal, nasal; and the pulmonary system.

56. The method as claimed in claim 55, wherein the pharmaceutical formulations are in the form selected from the group: gel; gel spray; tablet; liquid; capsule and for vaporisation.

57. The method as claimed in claim 26, wherein the cannabinoids are present as a cannabis based medicine extract (CBME).

58. The method as claimed in claim 26, wherein the combination of cannabinoids comprises:
a) a cannabis based medicinal extract which comprises THC at more than 90% of the total cannabinoid content in the extract; and
b) a cannabis based medicinal extract which comprises CBD at more than 90% of the total cannabinoid content in the extract.

59. The method as claimed in claim 26, wherein the cannabinoids are substantially pure.

60. The method as claimed in claim 26, wherein the cannabinoids are synthetic.

61. The method as claimed in claim 26, wherein the cannabinoids are administered in combination with one or more other medicinal substances.

62. The method as claimed in claim 61, wherein the cannabinoids are administered in addition to one or more analgesic drugs, one or more opiate or opiate related drugs, one or more anticonvulsant drugs and/or one or more antidepressant drugs.

63. The method as claimed in claim 61, wherein the cannabinoids are administered separately, simultaneously or sequentially to the one or more other drugs.

64. The method as claimed in claim 27, wherein the ratio of CBD:THC by weight is between 5:1 and 1:5.

65. The method as claimed in claim 27, wherein the ratio of CBD:THC by weight is between 2:1 and 1:2.

66. The method as claimed in claim 27, wherein the ratio of CBD:THC by weight is substantially 1:1.

67. The method as claimed in claim 66, wherein the ratio of CBD:THC by weight is 0.93:1.

68. The method as claimed in claim 27, wherein the cannabinoids are packaged for delivery in a titratable dosage form.

69. The method as claimed in claim 27, wherein the cannabinoid CBD is administered separately, simultaneously or sequentially to the cannabinoid THC.

70. The method as claimed in claim 27, wherein a unit dose taken by a patient is in the range of 5-25 mg of each cannabinoid.

71. The method as claimed in claim 27, wherein the maximum daily dosage dose of each cannabinoid is less than or equal to 120 mg of CBD and less than or equal to 130 mg of THC.

72. The method as claimed in claim 27, wherein the pharmaceutical formulations are packaged for delivery such that delivery is targeted to an area selected from the group: sublingual; buccal; oral; rectal, nasal; and the pulmonary system.

73. The method as claimed in claim 72, wherein the pharmaceutical formulations are in the form selected from the group: gel; gel spray; tablet; liquid; capsule and for vaporisation.

74. The method as claimed in claim 27, wherein the cannabinoids are present as a cannabis based medicine extract (CBME).

75. The method as claimed in claim 27, wherein the combination of cannabinoids comprises:
a) a cannabis based medicinal extract which comprises THC at more than 90% of the total cannabinoid content in the extract; and
b) a cannabis based medicinal extract which comprises CBD at more than 90% of the total cannabinoid content in the extract.

76. The method as claimed in claim 27, wherein the cannabinoids are substantially pure.

77. The method as claimed in claim 27, wherein the cannabinoids are synthetic.

78. The method as claimed in claim 27, wherein the cannabinoids are administered in combination with one or more other medicinal substances.

79. The method as claimed in claim 78, wherein the cannabinoids are administered in addition to one or more analgesic drugs, one or more opiate or opiate related drugs, one or more anticonvulsant drugs and/or one or more antidepressant drugs.

80. The method as claimed in claim 78, wherein the cannabinoids are administered separately, simultaneously or sequentially to the one or more other drugs.

81. The method as claimed in claim 28, wherein the ratio of CBD:THC by weight is between 5:1 and 1:5.

82. The method as claimed in claim 28, wherein the ratio of CBD:THC by weight is between 2:1 and 1:2.

83. The method as claimed in claim 28, wherein the ratio of CBD:THC by weight is substantially 1:1.

84. The method as claimed in claim 83, wherein the ratio of CBD:THC by weight is 0.93:1.

85. The method as claimed in claim 28, wherein the cannabinoids are packaged for delivery in a titratable dosage form.

86. The method as claimed in claim 28, wherein the cannabinoid CBD is administered separately, simultaneously or sequentially to the cannabinoid THC.

87. The method as claimed in claim 28, wherein a unit dose taken by a patient is in the range of 5-25 mg of each cannabinoid.

88. The method as claimed in claim 28, wherein the maximum daily dosage dose of each cannabinoid is less than or equal to 120 mg of CBD and less than or equal to 130 mg of THC.

89. The method as claimed in claim 28, wherein the pharmaceutical formulations are packaged for delivery such that delivery is targeted to an area selected from the group: sublingual; buccal; oral; rectal, nasal; and the pulmonary system.

90. The method as claimed in claim 89, wherein the pharmaceutical formulations are in the form selected from the group: gel; gel spray; tablet; liquid; capsule and for vaporisation.

91. The method as claimed in claim 28, wherein the cannabinoids are present as a cannabis based medicine extract (CBME).

92. The method as claimed in claim 28, wherein the combination of cannabinoids comprises:
a) a cannabis based medicinal extract which comprises THC at more than 90% of the total cannabinoid content in the extract; and
b) a cannabis based medicinal extract which comprises CBD at more than 90% of the total cannabinoid content in the extract.

93. The method as claimed in claim 28, wherein the cannabinoids are substantially pure.

94. The method as claimed in claim 28, wherein the cannabinoids are synthetic.

95. The method as claimed in claim 28, wherein the cannabinoids are administered in combination with one or more other medicinal substances.

96. The method as claimed in claim 95, wherein the cannabinoids are administered in addition to one or more analgesic drugs, one or more opiate or opiate related drugs, one or more anticonvulsant drugs and/or one or more antidepressant drugs.

97. The method as claimed in claim 95, wherein the cannabinoids are administered separately, simultaneously or sequentially to the one or more other drugs.
